(19) United States
(12) Patent Application Publication
(10) Pub. No.: US 2016/0345901 A1
(43) Pub. Date: Dec. 1, 2016

(54) Wearable Brain Activity Monitor

(71) Applicant: Robert A. Connor, Forest Lake, MN (US)

(72) Inventor: Robert A. Connor, Forest Lake, MN (US)

(73) Assignee: Medibotics LLC, Forest Lake, MN (US)

(21) Appl. No.: 15/236,401

(22) Filed: Aug. 13, 2016

Related U.S. Application Data
(63) Continuation-in-part of application No. 15/136,948, filed on Apr. 24, 2016.
Application No. 15/136,948 is a continuation-in-part of application No. 14/599,522, filed on Jan. 18, 2015.
Application No. 14/599,522 is a continuation-in-part of application No. 14/562,719, filed on Dec. 7, 2014.
Application No. 14/562,719 is a continuation-in-part of application No. 14/599,522, filed on Jan. 18, 2015.
(60) Provisional application No. 61/932,517, filed on Jan. 28, 2014.
(60) Provisional application No. 61/932,517, filed on Jan. 28, 2014.
(60) Provisional application No. 61/939,244, filed on Feb. 12, 2014.
(60) Provisional application No. 62/017,615, filed on Jun. 26, 2014.
(60) Provisional application No. 62/089,696, filed on Dec. 9, 2014.
(60) Provisional application No. 62/160,172, filed on May 12, 2015.
(60) Provisional application No. 62/169,661, filed on Jun. 2, 2015.
(60) Provisional application No. 62/303,126, filed on Mar. 3, 2016.
(60) Provisional application No. 62/322,594, filed on Apr. 14, 2016.

(51) Int. Cl. A61B 5/00; A61B 5/04

(52) U.S. Cl. CPC A61B 5/6803; A61B 5/04008; A61B 5/0006

(57) ABSTRACT

This invention is a wearable brain activity monitor with electromagnetic brain activity sensors which are held on a person's head by a partially-circumferential headband. The partially-circumferential headband curves around the lower-posterior surface of a person's head, from one ear to the other, and has forward ends which extend upward from the person's ears to the sides of the person's forehead.

### CROSS-REFERENCE TO RELATED APPLICATIONS

This patent application:

is a continuation-in-part of U.S. patent application Ser. No. 15/136,948 entitled “Wearable and Mobile Brain Computer Interface (BCI) Device and Method” by Robert A. Connor with a filing date of Apr. 24, 2016 which: (1) was a continuation-in-part of U.S. patent application Ser. No. 14/599,522 entitled “Mobile Wearable Electromagnetic Brain Activity Monitor” by Robert A. Connor with a filing date of Jan. 18, 2015 which: (1a) was a continuation in part of U.S. patent application Ser. No. 14/562,719 entitled “Willpower Glasses (™)—A Wearable Food Consumption Monitor” by Robert A. Connor with a filing date of Dec. 7, 2014 which claimed the priority benefit of U.S. Provisional Patent Application No. 61/932,517 entitled “Nutrode (™): Wearable EEG Monitor for Modifying Food Consumption” by Robert A. Connor with a filing date of Jan. 28, 2014; (1b) claimed the priority benefit of U.S. Provisional Patent Application No. 61/932,517 entitled “Nutrode (™): Wearable EEG Monitor for Modifying Food Consumption” by Robert A. Connor with a filing date of Jan. 28, 2014; (1c) claimed the priority benefit of U.S. Provisional Patent Application No. 61/939,244 entitled “Brainwave-Controlled Eyewear” by Robert A. Connor with a filing date of Feb. 12, 2014; (1d) claimed the priority benefit of U.S. Provisional Patent Application No. 62/017,615 entitled “Nervision (™) Integrated Eyewear and EEG Monitor” by Robert A. Connor with a filing date of Jun. 26, 2014; and (le) claimed the priority benefit of U.S. Provisional Patent Application No. 62/089,696 entitled “Electroencephalographic Eyewear” by Robert A. Connor with a filing date of Dec. 9, 2014; (2) claimed the priority benefit of U.S. Provisional Patent Application No. 62/160,172 entitled “Hair-Engaging Mobile Brain Activity Monitor” by Robert A. Connor with a filing date of May 12, 2015; (3) claimed the priority benefit of U.S. Provisional Patent Application No. 62/169,661 entitled “Internet of Thinks (IoT): A Brain Computer Interface (BCI) Using EEG Patterns Associated with the Same Command Across Different Action Modes” by Robert A. Connor with a filing date of Jun. 2, 2015; (4) claimed the priority benefit of U.S. Provisional Patent Application No. 62/303,126 entitled “Undulating Mobile EEG Monitor Spanning a Portion of the Forehead” by Robert A. Connor with a filing date of Mar. 3, 2016; and (5) claimed the priority benefit of U.S. Provisional Patent Application No. 62/322,594 entitled “Halo-Style Mobile Electroencephalographic (EEG) Monitor” by Robert A. Connor with a filing date of Apr. 14, 2016; and

is a continuation-in-part of U.S. patent application Ser. No. 14/599,522 entitled “Mobile Wearable Electromagnetic Brain Activity Monitor” by Robert A. Connor with a filing date of Jan. 18, 2015 which: (1) was a continuation in part of U.S. patent application Ser. No. 14/562,719 entitled “Willpower Glasses (™)—A Wearable Food Consumption Monitor” by Robert A. Connor with a filing date of Dec. 7, 2014 which claimed the priority benefit of U.S. Provisional Patent Application No. 61/932,517 entitled “Nutrode (™): Wearable EEG Monitor for Modifying Food Consumption” by Robert A. Connor with a filing date of Jan. 28, 2014; (2) claimed the priority benefit of U.S. Provisional Patent Application No. 61/932,517 entitled “Nutrode (™): Wearable EEG Monitor for Modifying Food Consumption” by Robert A. Connor with a filing date of Jan. 28, 2014; (3) claimed the priority benefit of U.S. Provisional Patent Application No. 61/939,244 entitled “Brainwave-Controlled Eyewear” by Robert A. Connor with a filing date of Feb. 12, 2014; (4) claimed the priority benefit of U.S. Provisional Patent Application No. 62/017,615 entitled “Nervision (™) Integrated Eyewear and EEG Monitor” by Robert A. Connor with a filing date of Jun. 26, 2014; and (5) claimed the priority benefit of U.S. Provisional Patent Application No. 62/089,696 entitled “Electroencephalographic Eyewear” by Robert A. Connor with a filing date of Dec. 9, 2014.

The entire contents of these related applications are incorporated herein by reference.

### FEDERALLY SPONSORED RESEARCH

Not Applicable

### SEQUENCE LISTING OR PROGRAM

Not Applicable

### BACKGROUND

Field Of Invention

This invention relates to wearable devices for measuring electromagnetic brain activity.

### INTRODUCTION

This invention relates to a mobile wearable brain activity monitor for measuring electromagnetic energy from a person's brain. The ability to measure electromagnetic brain activity (such as electroencephalographic EEG activity) with a mobile wearable device allows such measurement while a person is ambulatory. With a mobile and wearable device, a person is free to do their normal activities. This provides useful information which is not possible with EEG monitoring devices which require that the person stay in fixed location (such as a hospital or medical office).

### REVIEW AND CATEGORIZATION OF THE RELEVANT ART

It can be challenging trying to classify relevant art in this field into discrete categories. However, classification of relevant art into categories, even if imperfect, can be an invaluable tool for reviewing the relevant art. Towards this end, I herein identify 12 categories of relevant art and provide examples of relevant art in each category (including patent or patent application number, inventor, publication date, and title). Some examples of relevant art disclose multiple concepts and thus appear in more than one category.

The 12 categories of relevant art which are used for this review are as follows: (1) device with [multiple] front-to-back arcuate members and EEG/brainwave sensors; (2) device with [multiple] side-to-side arcuate members and EEG/brainwave sensors; (3) device with multiple cross-crossing arcuate members and EEG/brainwave sensors; (4) device with multiple arms radially-extending from side and EEG/brainwave sensors; (5) device with multiple arms radially-downward from top and EEG/brainwave sensors; (6) device with multiple arms radially-forward from rear and EEG/brainwave sensors; (7) device with multiple arms radially-backward from front and EEG/brainwave sensors; (8) device with circular horizontal loop (e.g. headband style) and EEG/brainwave sensors; (9) device with top semicircular loop (e.g. headphone style) and EEG/brainwave sensors; (10) device with rear semicircular loop and EEG/brainwave sensors; (11) device with frontal semicircular loop and EEG/brainwave sensors; and (12) device like eyeglasses or other eyewear with EEG/brainwave sensors. Of these, category (10) is probably the most relevant to this invention.

I have labeled this section as a review of the relevant art, instead of a review of the prior art, for two reasons. First, some of the art included in this review has a priority date after the priority date of this disclosure, so I do not wish to call all of this art “prior.” Second, some of the examples in this present disclosure can be classified into one or more of these categories but are nonetheless novel, so I do not wish to imply that all of the art in these categories is “prior”. These caveats notwithstanding, I hope that the reader finds this review and categorization of the relevant art to be useful.

1. Device with [Multiple] Front-to-Back Arcuate Member(s) and EEG/Brainwave Sensor(s)

Devices in this category hold electromagnetic brain activity sensors in contact with (or proximity to) a person's head using (multiple) arcing member(s) which span a person's head from front-to-back (or vice versa). Devices in this category can look similar to some types of bicycle helmets with front-to-back arcuate members. In an example, the front-to-back arcing members can converge at the forehead and at the rear of the head. In an example, a device in this category can comprise: a first arcuate member which encircles a person's head: a second arcuate member which loops front-to-back over the top of the head; and third and fourth arcuate members which loop front-to-back over the sides of the head between the first and second members. Devices in this category can hold a relatively large number of electromagnetic brain activity sensors along arcuate front-to-rear lines on a person's head. However, such devices tend to be too obtrusive to wear during the activities of daily life.

Relevant art which appears to be within this category includes U.S. patents: U.S. Pat. No. 3,998,213 (Price, Dec. 21, 1976, “Self-Adjustable Holder for Automatically Positioning Electroencephalographic Electrodes”), U.S. Pat. No. 8,355,769 (Levendowski et al., Jan. 15, 2013, “System for the Assessment of Sleep Quality in Adults and Children”), U.S. Pat. No. 8,463,354 (Fadem, Jun. 11, 2013, “Electrode System with Rigid-Flex Circuit”), U.S. Pat. No. 8,639,313 (Westbrook et al, Jan. 28, 2014, “System for the Assessment of Sleep Quality in Adults and Children”); and U.S. patent applications 20100125190 (Fadem, May 20, 2010, “Electrode System”), 20100240982 (Westbrook et al., Sep. 23, 2010, “System for the Assessment of Sleep Quality in Adults and Children”), and 20130131464 (Westbrook et al., May 23, 2013, “System for the Assessment of Sleep Quality in Adults and Children”).

2. Device with [Multiple] Side-to-Side Arcuate Member(s) and EEG/Brainwave Sensor(s)

Devices in this category hold electromagnetic brain activity sensors in contact with (or proximity to) a person's head using (multiple) arcing member(s) which span a person's head from side to side. In an example, side-to-side arcing members can converge near, or over, the person's ears. In an example, devices in this category can be similar to those in the previous category, except having been rotated 90 degrees so that the arcuate members converge on the sides of the person's head rather than the front and rear of the person's head. Devices in this category can hold a relatively large number of electromagnetic brain activity sensors along arcuate side-to-side lines on a person's head. However, such devices tend to be too obtrusive to wear during the activities of daily life.

Relevant art which appears to be within this category includes U.S. patents: U.S. Pat. No. 4,836,219 (Hobson et al., Jun. 6, 1989, “Electronic Sleep Monitor Headgear”), U.S. Pat. No. 5,800,351 (Mann, Sep. 1, 1998, “Electrode Supporting Head Set”), U.S. Pat. No. 6,574,513 (Collura et al., Jun. 3, 2003, “EEG Electrode Assemblies”), U.S. Pat. No. 7,158,822 (Payne Jr., Jan. 2, 2007, “Electrode Holder, Headwear, and Wire Jacket Adapted for Use in Sleep Apnea Testing”), and U.S. Pat. No. 7,885,706 (Ludvig et al., Feb. 8, 2011, “System and Device for Seizure Detection”).

Relevant art which appears to be within this category also includes U.S. patent applications: 20030018278 (Jordan, Jan. 23, 2003, “Electroencephalogram Acquisition Unit and System”), 20050277821 (Payne, Dec. 15, 2005, “Electrode Holder, Headwear, and Wire Jacket Adapted for Use in Sleep Apnea Testing”), 20070112262 (Payne, May 17, 2007, “Electrode Holder, Headwear, and Wire Jacket Adapted for Use in Sleep Apnea Testing”), 20080082019 (Ludving et al., Apr. 3, 2008, “System and Device for Seizure Detection”), 20090281446 (Ludvig et al., Nov. 12, 2009, “System and Device for Seizure Detection”), 20110015503 (Joffe et al., Jan. 20, 2011, “Medical Apparatus for Collecting Patient Electroencephalogram (EEG) Data”), and 20110270117 (Warwick et al., Nov. 3, 2011, “Remote Continuous Seizure Monitor and Alarm”).

3. Device with Multiple Cross-Crossing Arcuate Members and EEG/Brainwave Sensor(s)

Devices in this category hold electromagnetic brain activity sensors in contact with (or proximity to) a person's head using multiple arcing members which span a person's head from front-to-rear and also multiple arcing members which span a person's head from side-to-side. In an example, the front-to-rear arcuate members and the side-to-side arcuate members can form a criss-cross pattern on the person's head. Devices in this category can hold a relatively large number of electromagnetic brain activity sensors on a person's head. However, such devices tend to be too obtrusive to wear during the activities of daily life.

Relevant art which appears to be within this category includes U.S. patents: U.S. Pat. No. 3,998,213 (Price, Dec. 21, 1976, “Self-Adjustable Holder for Automatically Positioning Electroencephalographic Electrodes”), U.S. Pat. No. 5,293,867 (Oommen, Mar. 15, 1994, “Method and Apparatus for Marking Electrode Locations for Electroencephalographic Procedure”), U.S. Pat. No. 5,479,934 (Imran, Jan. 2, 1996, “EEG Headpiece with Disposable Electrodes and Apparatus and System and Method for Use Therewith”), U.S. Pat. No. 6,488,617 (Katz, Dec. 3, 2002, “Method and Device for Producing a Desired Brain State”), U.S. Pat. No. 8,463,354 (Fadem, Jun. 11, 2013, “Electrode System with Rigid-Flex Circuit”); and U.S. patent applications 20030018278 (Jordan, Jan. 23, 2003, “Electroencephalogram Acquisition Unit and System”), and 20100125190 (Fadem, May 20, 2010, “Electrode System”).

4. Device with Multiple Arms Radially-Extending from Side and EEG/Brainwave Sensor(s)

Devices in this category hold electromagnetic brain activity sensors in contact with (or proximity to) a person's head using multiple sensor-holding protrusions, fingers, or arms which extend radially outward from a central position on one side (or from central positions on both sides) of a person's head. In an example, such devices can include bilateral clusters (one on each side of the head) of radially-extending protrusions, fingers, or arms. In an example, radially-extending protrusions, fingers, or arms can curve around the head toward the front, top, and/or rear portions of the head. To use colorful language, some such devices can look like a wearer has one or two starfish (or even octopi) clinging to the sides of their head. Such devices can be less obtrusive than those in the preceding categories (especially when they do not span the forehead or the top of the head), but can still attract attention if worn during the activities of daily life.

Relevant art which appears to be within this category includes U.S. patents: U.S. Pat. No. 5,954,667 (Finkenzeller et al., Sep. 21, 1999, “Device for Deriving Acoustically Evoked Brain Potentials”), U.S. Pat. No. 8,271,075 (Chuang et al., Sep. 18, 2012, “Audio Headset with Bio-Signal Sensors”), U.S. Pat. No. 8,392,250 (Pradeep et al., Mar. 5, 2013, “Neuro-Response Evaluated Stimulus in Virtual Reality Environments”), U.S. Pat. No. 8,392,251 (Pradeep et al., Mar. 5, 2013, “Location Aware Presentation of Stimulus Material”), U.S. Pat. No. 8,396,744 (Pradeep et al., Mar. 12, 2013, “Effective Virtual Reality Environments for Presentation of Marketing Materials”), U.S. Pat. No. 8,548,852 (Pradeep et al., Oct. 1, 2013, “Effective Virtual Reality Environments for Presentation of Marketing Materials”), and U.S. Pat. No. 8,655,428 (Pradeep et al., Feb. 18, 2014, “Neuro-Response Data Synchronization”).

Relevant art which appears to be within this category also includes U.S. patent applications: 20070106169 (Fadem, May 10, 2007, “Method and System for an Automated E.E.G. System for Auditory Evoked Responses”), 20070191727 (Fadem, Aug. 16, 2007, “Evoked Response Testing System for Neurological Disorders”), 20070225585 (Washbon and Delic, Sep. 27, 2007, “Headset for Electrodes”), 20070238945 (Delic et al., Oct. 11, 2007, “Electrode Headset”), 20080208072 (Fadem et al., Aug. 28, 2008, “Biopotential Waveform Data Fusion Analysis and Classification Method”), 20110237971 (Pradeep et al., Sep. 29, 2011, “Discrete Choice Modeling Using Neuro-Response Data”), and 20110282231 (Pradeep et al., Nov. 17, 2011, “Mechanisms for Collecting Electroencephalography Data”).

Relevant art which appears to be within this category also includes U.S. patent applications: 20110282232 (Pradeep et al., Nov. 17, 2011, “Neuro-Response Data Synchronization”), 20120072289 (Pradeep et al., Mar. 22, 2012, “Biometric Aware Content Presentation”), 20130131537 (Tam, May 23, 2013, “Tong Ren Brainwave Entrainment”), 20130185144 (Pradeep et al., Jul. 18, 2013, “Systems and Methods for Analyzing Neuro-Response Data and Virtual Reality Environments”), 20130314243 (Le, Nov. 28, 2013, “System and Method for Enabling Collaborative Analysis of a Biosignal”), 20130317382 (Le, Nov. 28, 2013, “System and Method for Providing and Aggregating Biosignals and Action Data”), and 20130317384 (Le, Nov. 28, 2013, “System and Method for Instructing a Behavior Change in a User”).

5. Device with Multiple Arms Radially-Downward from Top and EEG/Brainwave Sensor(s)

Devices in this category hold electromagnetic brain activity sensors in contact with (or proximity to) a person's head using multiple sensor-holding protrusions, fingers, or arms which extend radially downward from a position on the top of a person's head. In an example, radially-extending protrusions, fingers, or arms can curve around the head toward the front, sides, and/or rear portions of the head. To use the colorful language from the previous category, now a figurative starfish (or octopus) is clinging to the top of the person's head. Such devices can be less obtrusive than some of those in the preceding categories, but can still attract attention if worn during the activities of daily life.

Relevant art which appears to be within this category includes U.S. patents: U.S. Pat. No. 6,067,464 (Musha, May 23, 200, “Electrode”), U.S. Pat. No. 6,154,669 (Hunter et al., Nov. 28, 2000, “Headset for EEG Measurements”), U.S. Pat. No. 6161030 (Levendowski et al., Dec. 12, 2000, “Portable EEG Electrode Locator Headgear”), U.S. Pat. No. 6,381,481 (Levendowski et al., Apr. 30, 2002, “Portable EEG Electrode Locator Headgear”), U.S. Pat. No. 7,551,952 (Gevins et al., Jun. 23, 2009, “EEG Electrode Headset”), U.S. Pat. No. 8,103,328 (Turner et al., Jan. 24, 2012, “Self-Locating Sensor Mounting Apparatus”), U.S. Pat. No. 8,392,250 (Pradeep et al., Mar. 5, 2013, “Neuro-Response Evaluated Stimulus in Virtual Reality Environments”), U.S. Pat. No. 8,392,251 (Pradeep et al., Mar. 5, 2013, “Location Aware Presentation of Stimulus Material”), U.S. Pat. No. 8,396,744 (Pradeep et al., Mar. 12, 2013, “Effective Virtual Reality Environments for Presentation of Marketing Materials”), U.S. Pat. No. 8,548,852 (Pradeep et al., Oct. 1, 2013, “Effective Virtual Reality Environments for Presentation of Marketing Materials”), and U.S. Pat. No. 8,655,428 (Pradeep et al., Feb. 18, 2014, “Neuro-Response Data Synchronization”).

Relevant art which appears to be within this category also includes U.S. patent applications: 20020029005 (Levendowski et al., Mar. 7, 2002, “Portable EEG Electrode Locator Headgear”), 20070093706 (Gevins et al., Apr. 26, 2007, “EEG Electrode Headset”), 20090088619 (Turner et al., Apr. 2, 2009, “Self-Locating Sensor Mounting Apparatus”), 20110098593 (Low et al., Apr. 28, 2011, “Head Harness & Wireless EEG Monitoring System”), 20110237971 (Pradeep et al., Sep. 29, 2011, “Discrete Choice Modeling Using Neuro-Response Data”), 20110282231 (Pradeep et al., Nov. 17, 2011, “Mechanisms for Collecting Electroencephalography Data”), 20110282232 (Pradeep et al., Nov. 17, 2011, “Neuro-Response Data Synchronization”), 20120072289 (Pradeep et al., Mar. 22, 2012, “Biometric Aware Content Presentation”), and 20130185144 (Pradeep et al., Jul. 18, 2013, “Systems and Methods for Analyzing Neuro-Reponse Data and Virtual Reality Environments”).

6. Device with Multiple Arms Radially-Forward from Rear and EEG/Brainwave Sensor(s)

Devices in this category hold electromagnetic brain activity sensors in contact with (or proximity to) a person's head using multiple sensor-holding protrusions, fingers, or arms which extend radially forward from a central position at the rear of a person's head. In an example, radially-extending protrusions, fingers, or arms can curve around the head toward the top and sides of the head. To use the colorful language from the previous category, now a figurative starfish (or octopus) is clinging to the back of the person's head. Such devices can be less obtrusive than some of those in the preceding categories, but can still attract attention if worn during the activities of daily life.

Relevant art which appears to be within this category includes U.S. patents: U.S. Pat. No. 4770180 (Schmidt et al., Sep. 13, 1988, “Electroencephalographic Head Set with a Disposable Monitor”), U.S. Pat. No. 4967038 (Gevins et al., Oct. 30, 1990, “Dry Electrode Brain Wave Recording System”), U.S. Pat. No. 5038782 (Gevins et al., Aug. 13, 1991, “Electrode System for Brain Wave Detection”), and D565735 (Washbon, Apr. 1, 2008, “Electrode Headset”); and U.S. patent applications 20070225585 (Washbon and Delic, Sep. 27, 2007, “Headset for Electrodes”), 20070238945 (Delic et al., Oct. 11, 2007, “Electrode Headset”), 20090105576 (Do et al., Apr. 23, 2009, “Electrode Conductive Element”), 20120029379 (Sivadas, Feb. 2, 2012, “Mind Strength Trainer”), and 20130046206 (Preminger, Feb. 21, 2013, “System and Method for Neurocognitive Training and/or Neuropsychological Assessment”).

7. Device with Multiple Arms Radially-Backward from Front and EEG/Brainwave Sensor(s)

Devices in this category hold electromagnetic brain activity sensors in contact with (or proximity to) a person's head using multiple sensor-holding protrusions, fingers, or arms which extend radially backward from a position on the front of a person's head (such as the forehead). In an example, radially-extending protrusions, fingers, or arms can curve around the head toward the top and sides of the head. Such devices can be obtrusive and attract attention, especially if worn to a showing of the movie “Aliens”. Relevant art which appears to be within this category includes U.S. patent application 20020188216 (Kayyali et al., Dec. 12, 2002, “Head Mounted Medical Device”).

8. Device with Circular Horizontal Loop (e.g. Headband Style) and EEG/Brainwave Sensor(s)

Devices in this category hold electromagnetic brain activity sensors in contact with (or proximity to) a person's head using a sensor-holding member which is configured like a headband, ring, or other generally-circular member which encircles a person's head in (or close to) a horizontal plane when the person is upright. In an example, such a device can span a portion of a person's forehead as it encircles the person's head. Since devices in this category can span a potion of the forehead, such devices can be used with sensors which require contact with (or proximity to) portions of the head which do not have hair Such devices can be appropriate for wearing while running or doing other types of exercise, but there are still many settings wherein wearing a headband or head-encircling ring is generally not appropriate.

Relevant art which appears to be within this category includes U.S. patents: U.S. Pat. No. 6,001,065 (Devito, Dec. 14, 1999, “Method and Apparatus for Measuring and Analyzing Physiological Signals for Active or Passive Control of Physical and Virtual Spaces and the Contents Therein”), U.S. Pat. No. 6,171,258 (Karakasoglu et al., Jan. 9, 2001, “Multi-Channel Self-Contained Apparatus and Method for Diagnosis of Sleep Disorders”), U.S. Pat. No. 6,254,536 (Devito, Jul. 3, 2001, “Method and Apparatus for Measuring and Analyzing Physiological Signals for Active or Passive Control of Physical and Virtual Spaces and the Contents Therein”), U.S. Pat. No. 6,811,538 (Westbrook et al., Nov. 2, 2004, “Sleep Apnea Risk Evaluation”), U.S. Pat. No. 7,297,119 (Westbrook et al., Nov. 20, 2007, “Sleep Apnea Risk Evaluation”), and U.S. Pat. No. 7,885,706 (Ludvig et al., Feb. 8, 2011, “System and Device for Seizure Detection”).

Relevant art which appears to be within this category also includes U.S. patent applications: 20010056225 (DeVito, Dec. 27, 2001, “Method and Apparatus for Measuring and Analyzing Physiological Signals for Active or Passive Control of Physical and Virtual Spaces and the Contents Therein”), 20020165462 (Westbrook et al., Nov. 7, 2002, “Sleep Apnea Risk Evaluation”), 20020188216 (Kayyali et al., Dec. 12, 2002, “Head Mounted Medical Device”), 20040267152 (Pineda, Dec. 20, 2004, “Method and System for Predicting and Preventing Seizures”), 20050027207 (Westbrook et al., Feb. 3, 2005, “Sleep Apnea Risk Evaluation”), and 20070249952 (Rubin et al., Oct. 25, 2007, “Systems and Methods for Sleep Monitoring”).

Relevant art which appears to be within this category also includes U.S. patent applications: 20080082019 (Ludving et al., Apr. 3, 2008, “System and Device for Seizure Detection”), 20090281446 (Ludvig et al., Nov. 12, 2009, “System and Device for Seizure Detection”), 20100099954 (Dickinson et al., Apr. 22, 2010, “Data-Driven Sleep Coaching System”), 20120150545 (Simon, Jun. 14, 2012, “Brain-Computer Interface Test Battery for the Physiological Assessment of Nervous System Health”), 20130060097 (Rubin, Mar. 7, 2013, “Multi-Modal Sleep System”), 20130127708 (Jung et al., May 23, 2013, “Cell-Phone Based Wireless and Mobile Brain-Machine Interface”), and 20130338446 (Van Vugt et al., Dec. 19, 2013, “Sleep Disturbance Monitoring Apparatus”).

9. Device with Top Semicircular Loop (e.g. Headphone Style) and EEG/Brainwave Sensor(s)

Devices in this category hold electromagnetic brain activity sensors in contact with (or proximity to) a person's head using a (semicircular) arcuate member which looks like a set of headphones, hair band, or tiara. In an example, such a device can loop over the top of a person's head, from one side to the other side. In an example, such a device can loop over the top of a person's head from one ear to the other ear. In example, such a device can not only look like a set of headphones, but can actually be a set of headphones, wherein these headphones also include one or more electromagnetic brain activity sensors. Wearing a set of headphones or a hair band is more common (and thus may attract less attention) than wearing most of the devices discussed in preceding categories, but there are still many settings wherein wearing such a device would attract attention and be inappropriate.

Relevant art which appears to be within this category includes U.S. patents: U.S. Pat. No. 4,697,598 (Bernard et al., Oct. 6, 1987, “Evoked Potential Autorefractometry System”), U.S. Pat. No. 4,709,702 (Sherwin, Dec. 1, 1987, “Electroencephalographic Cap”), U.S. Pat. No. 5,740,812 (Cowan, Apr. 21, 1998, “Apparatus for and Method of Providing Brainwave Biofeedback”), U.S. Pat. No. 6,154,669 (Hunter et al., Nov. 28, 2000, “Headset for EEG Measurements”), U.S. Pat. No. 6,167,298 (Levin, Dec. 26, 2000, “Devices and Methods for Maintaining an Alert State of Consciousness Through Brain Wave Monitoring”), U.S. Pat. No. 7,689,274 (Mullen et al., Mar. 30, 2010, “Brain-Wave Aware Sleep Management”), U.S. Pat. No. 8,271,075 (Chuang et al., Sep. 18, 2012, “Audio Headset with Bio-Signal Sensors”), and U.S. Pat. No. 8,301,218 (Nguyen et al., Oct. 30, 2012, “Contoured Electrode”), U.S. Pat. No. 8,812,075 (Nguyen et al., Aug. 19, 2014, “Contoured Electrode”).

Relevant art which appears to be within this category also includes U.S. patent applications: 20120029379 (Sivadas, Feb. 2, 2012, “Mind Strength Trainer”), 20120226127 (Asjes et al., Sep. 6, 2012, “Device for Positioning Electrodes on a User's Scalp”), 20130177883 (Barnehama et al., Jul. 11, 2013, “Systems and Methods for Directing Brain Activity”), and 20130310676 (Jung, Nov. 21, 2013, “EEG Hair Band”).

10. Device with Rear Semicircular Loop and EEG/Brainwave Sensor(s)

Devices in this category hold electromagnetic brain activity sensors in contact with (or proximity to) a person's head using a (semicircular) arcuate member which loops around the rear portion of a person's head, from one side to the other side. In an example, such a device can loop around the rear portion of a person's head from one ear to the other ear. Such a device can be less obtrusive than many of the devices in preceding categories because it does not span the top of the head or face, but it is not well-suited for use with sensors which require contact with skin without hair. Relevant art which appears to be within this category includes U.S. patent application 20140316230 (Denison et al., Oct. 23, 2014, “Methods and Devices for Brain Activity Monitoring Supporting Mental State Development and Training”).

11. Device with Frontal Semicircular Loop and EEG/Brainwave Sensor(s)

Devices in this category hold electromagnetic brain activity sensors in contact with (or proximity to) a person's head using a (semicircular) arcuate member which loops around the front of a person's head, from one side to the other side. In an example, such a device can loop around the front of a person's head from one ear to the other ear. In an example, such a device can span a person's forehead. Such a device can be well-suited for use with sensors which require contact with skin without hair, but can be somewhat obtrusive since it spans a portion of a person's face. Relevant art which appears to be within this category includes U.S. patent application 20080177197 (Lee et al., Jul. 24, 2008, “Method and Apparatus for Quantitatively Evaluating Mental States Based on Brain Wave Signal Processing System”).

12. Device like Eyeglasses or other Eyewear with EEG/Brainwave Sensor(s)

Devices in this category hold electromagnetic brain activity sensors in contact with (or proximity to) a person's head using a sensor-holding member which looks like a pair of eyeglasses, goggles, or other eyewear. In an example, such a device can span from one ear, to the face, across the face (over the bridge of the nose), and then to the other ear. In example, such a device can not only look like a pair of eyeglasses, but can actually be a pair of eyeglasses, wherein these eyeglasses include one or more electromagnetic brain activity sensors. Some of the art in this category predominantly focuses on the optical aspects of a pair of eyeglasses, with only tangential mention of a possible EEG sensor, but such art is included in this category for the sake of completeness. Wearing a pair of eyeglasses is very common and thus attracts less attention than virtually all of the devices discussed in preceding categories. However, conventional eyeglass frames (especially those with straight side pieces) do not contact a person's temple or forehead. Accordingly, conventional eyeglass frame configurations are not ideally-suited for holding one or more electromagnetic brain activity sensors in contact with a person's temple and/or forehead.

Relevant art which appears to be within this category includes U.S. patents: U.S. Pat. No. 7,344,244 (Goodall et al., Mar. 18, 2008, “Adjustable Lens System with Neural-Based Control”), U.S. Pat. No. 7,390,088 (Goodall et al., Jun. 24, 2008, “Adjustable Lens System with Neural-Based Control”), U.S. Pat. No. 7,486,988 (Goodall et al., Feb. 3, 2009, “Method and System for Adaptive Vision Modification”), U.S. Pat. No. 8,244,342 (Goodall et al., Aug. 14, 2012, “Method and System for Adaptive Vision Modification”), U.S. Pat. No. 8,346,354 (Hyde et al., Jan. 1, 2013, “Determining a Neuromodulation Treatment Regimen in Response to Contactlessly Acquired Information”), U.S. Pat. No. 8,467,133 (Miller, Jun. 18, 2013, “See-Through Display with an Optical Assembly Including a Wedge-Shaped Illumination System”), U.S. Pat. No. 8,472,120 (Border et al., Jun. 25, 2013, “See-Through Near-Eye Display Glasses with a Small Scale Image Source”), U.S. Pat. No. 8,477,425 (Border et al., Jul. 2, 2013, “See-Through Near-Eye Display Glasses Including a Partially Reflective, Partially Transmitting Optical Element”), U.S. Pat. No. 8,482,859 (Border et al., Jul. 9, 2013, “See-Through Near-Eye Display Glasses Wherein Image Light Is Transmitted to and Reflected From an Optically Flat Film”), U.S. Pat. No. 8,488,246 (Border et al., Jul. 16, 2013, “See-Through Near-Eye Display Glasses Including a Curved Polarizing Film in the Image Source, a Partially Reflective, Partially Transmitting Optical Element and an Optically Flat Film”), and U.S. Pat. No. 8,562,540 (Goodall et al., Oct. 22, 2013, “Method and System for Adaptive Vision Modification”).

Relevant art which appears to be within this category also includes U.S. patent applications: 20060252978 (Vesely et al., Nov. 9, 2006, “Biofeedback Eyewear System”), 20060252979 (Vesely et al., Nov. 9, 2006, “Biofeedback Eyewear System”), 20070010757 (Goodall et al., Jan. 11, 2007, “Method and System for Adaptive Vision Modification”), 20070019279 (Goodall et al., Jan. 25, 2007, “Adjustable Lens System with Neural-Based Control”), 20070106145 (Kim et al., May 10, 2007, “Accessories for Remote Monitoring”), 20080161673 (Goodall et al., Jul. 3, 2008, “Method and System for Adaptive Vision Modification”), 20110028798 (Hyde et al., Feb. 3, 2011, “Electronically Initiating an Administration of a Neuromodulation Treatment Regimen Chosen in Response to Contactlessly Acquired Information”), 20110029038 (Hyde et al., Feb. 3, 2011, “Determining a Neuromodulation Treatment Regimen in Response to Contactlessly Acquired Information”), 20110029044 (Hyde et al., Feb. 3, 2011, “Stimulating a Nervous System Component of a Mammal in Response to Contactlessly Acquired Information”), 20110221656 (Haddick et al., Sep. 15, 2011, “Displayed Content Vision Correction with Electrically Adjustable Lens”), and 20110221669 (Shams et al., Sep. 15, 2011, “Gesture Control in an Augmented Reality Eyepiece”).

Relevant art which appears to be within this category also includes U.S. patent applications: 20110221672 (Osterhout et al., Sep. 15, 2011, “Hand-Worn Control Device in an Augmented Reality Eyepiece”), 20110222745 (Osterhout et al., Sep. 15, 2011, “Method and Apparatus for Biometric Data Capture”), 20110227820 (Haddick et al., Sep. 22, 2011, “Lock Virtual Keyboard Position in an Augmented Reality Eyepiece”), 20120062445 (Haddick et al., Mar. 15, 2012, “Adjustable Wrap Around Extendable Arm for a Head-Mounted Display”), 20120075168 (Osterhout et al., Mar. 29, 2012, “Eyepiece with Uniformly Illuminated Reflective Display”), 20120150545 (Simon, Jun. 14, 2012, “Brain-Computer Interface Test Battery for the Physiological Assessment of Nervous System Health”), 20120212398 (Border et al., Aug. 23, 2012, “See-Through Near-Eye Display Glasses Including a Partially Reflective, Partially Transmitting Optical Element”), and 20120212400 (Border et al., Aug. 23, 2012, “See-Through Near-Eye Display Glasses Including a Curved Polarizing Film in the Image Source, a Partially Reflective, Partially Transmitting Optical Element and an Optically Flat Film”).

Relevant art which appears to be within this category also includes U.S. patent applications: 20120218172 (Border et al., Aug. 30, 2012, “See-Through Near-Eye Display Glasses with a Small Scale Image Source”), 20120218301 (Miller, Aug. 30, 2012, “See-Through Display with an Optical Assembly Including a Wedge-Shaped Illumination System”), 20120235883 (Border et al., Sep. 20, 2012, “See-Through Near-Eye Display Glasses with a Light Transmissive Wedge Shaped Illumination System”), 20120235886 (Border et al., Sep. 20, 2012, “See-Through Near-Eye Display Glasses with a Small Scale Image Source”), 20120235887 (Border et al., Sep. 20, 2012, “See-Through Near-Eye Display Glasses Including a Partially Reflective, Partially Transmitting Optical Element and an Optically Flat Film”), and 20120235900 (Border et al., Sep. 20, 2012, “See-Through Near-Eye Display Glasses with a Fast Response Photochromic Film System for Quick Transition From Dark to Clear”).

Relevant art which appears to be within this category also includes U.S. patent applications: 20120236030 (Border et al., Sep. 20, 2012, “See-Through Near-Eye Display Glasses Including a Modular Image Source”), 20120242678 (Border et al., Sep. 27, 2012, “See-Through Near-Eye Display Glasses Including an Auto-Brightness Control for the Display Brightness Based on the Brightness in the Environment”), 20120242698 (Haddick et al., Sep. 27, 2012, “See-Through Near-Eye Display Glasses with a Multi-Segment Processor-Controlled Optical Layer”), 20130056010 (Walker et al., Mar. 7, 2013, “Autonomous Positive Airway Pressure System”), 20130127980 (Haddick et al., May 23, 2013, “Video Display Modification Based on Sensor Input for a See-Through Near-to-Eye Display”), and 20130242262 (Lewis, Sep. 19, 2013, “Enhanced Optical and Perceptual Digital Eyewear”).

Relevant art which appears to be within this category also includes U.S. patent applications: 20130303837 (Berka et al., Nov. 14, 2013, “Systems and Methods for Optimization of Sleep and Post-Sleep Performance”), 20130314303 (Osterhout et al., Nov. 28, 2013, “AR Glasses with User Action Control of and Between Internal and External Applications with Feedback”), 20140023999 (Greder, Jan. 23, 2014, “Detection and Feedback of Information Associated with Executive Function”), 20140267005 (Urbach, Sep. 18, 2014, “Eye Piece for Augmented and Virtual Reality”), 20140267401 (Urbach, Sep. 18, 2014, “Visual Cortex Thought Detector Interface”), 20140347265 (Aimone et al., Nov. 27, 2014, “Wearable Computing Apparatus and Method”), and 20140375545 (Ackerman et al., Dec. 25, 2014, “Adaptive Event Recognition”).

### SUMMARY OF THE INVENTION

This invention is a wearable brain activity monitoring device with a plurality of electromagnetic brain activity sensors which are held in place at selected locations on a person's head by a partially-circumferential headband. The partially-circumferential headband curves around the lower-posterior surface of a person's head, from one ear to the other, and has forward ends which extend upward from the person's ears to the sides of the person's forehead. In an example, the device can have six brain activity sensors which are located at the F3, F4, P3, P4, O1, and O2 standard EEG sensor locations. This enables ambulatory monitoring of person's electromagnetic brain activity in a relatively non-intrusive manner.

### INTRODUCTION TO THE FIGURES

FIG. 1 shows a first example of a wearable brain activity monitoring device with a plurality of electromagnetic brain activity sensors which are held in place at selected locations on a person's head by a partially-circumferential headband—with a control unit in front of the person's ear.

FIG. 2 shows a second example of a wearable brain activity monitoring device with a plurality of electromagnetic brain activity sensors which are held in place at selected locations on a person's head by a partially-circumferential headband—with an ear-perimeter engaging member.

FIG. 3 shows a first example of a wearable brain activity monitoring device with a plurality of electromagnetic brain activity sensors which are held in place at selected locations on a person's head by a partially-circumferential headband—with a data transmitter and/or receiver, data processor, and power source.

### DETAILED DESCRIPTION OF THE FIGURES

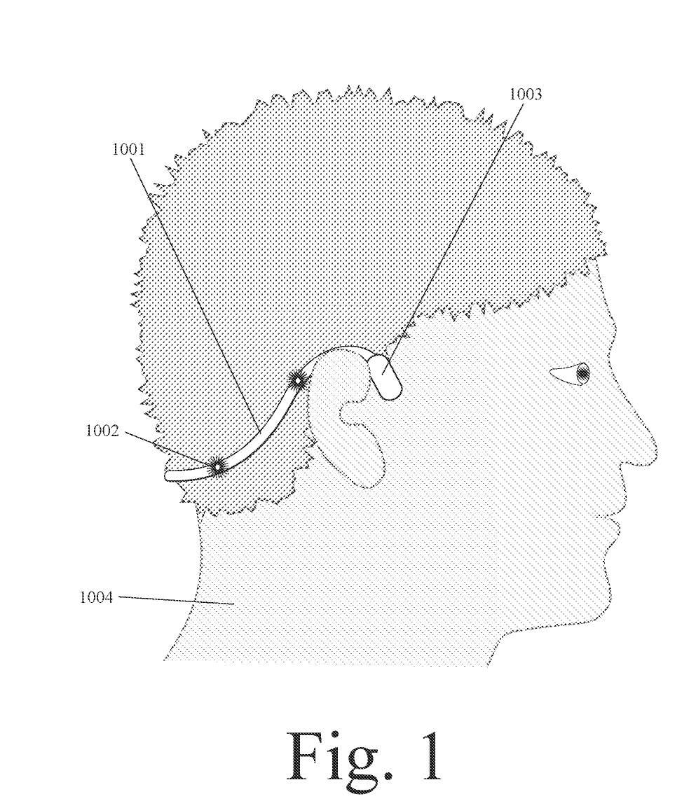

FIG. 1 shows a side view of an example of a wearable brain activity monitor comprising a head-worn sensor-positioning member 1001 which is configured to position a plurality of electrodes or other brain activity sensors, including 1002, at selected locations on a person's 1004 head. In this example, the sensor-positioning member is assumed to be substantially symmetric with respect to the right side (shown) and the left side (not shown) of the person's head. This monitor further comprises control unit 1003, which need not be replicated on the other side. In this example, sensor-positioning member 1001 comprises a loop that spans from one ear to the other, looping around the lower-posterior portion of the person's head. In an example, the average height of this loop is equal to, or lower than, the average height of the person's ears. In this example, the left-side and right-side ends of the loop curve around and hook over the tops of the person's left and right ears, respectively, terminating in locations forward of the upper portions of the ears. In this example, control unit 1003 is just forward of the upper portion of the left ear. In this example, the loop spans a lower portion of the person's temporal lobe and a portion of their cerebellum.

In an example, control unit 1003 can further comprise: a data processing component and a power source (or transducer). In an example, control unit 1003 can further comprise: a data processing component; a power source (or transducer); and a data transmitting (and receiving) component. In an example, control unit 1003 can be in wireless communication with an external (or remote) device and/or with another component of an overall system for monitoring brain activity. In an example, control unit 1003 can further comprise: a data processing component; a power source (or transducer); a data transmitting (and receiving) component; and a user interface. In an example, control unit 1003 can be physically connected to the array of electrodes (or other brain activity sensors) by wires or other electromagnetically-conductive pathways. In an example, control unit 1003 can be in wireless electromagnetic communication with an array of electrodes (or other brain activity sensors).

FIG. 1 also shows a wearable brain activity monitoring device comprising: a plurality of brain activity sensors (including 1002); a head-worn loop 1001 which is configured to span from one ear to the other around the lower-posterior portion of the head of a person 1004 wearing the loop, wherein the loop is configured to position the plurality of brain activity sensors at selected locations on the person's head; and a control unit 1003. In an example, the control unit can further comprise a data processing component and a power source or transducer. Relevant embodiment variations discussed elsewhere in this disclosure or in other disclosures which are incorporated by reference in the priority claims can be applied to the example shown here in this figure.

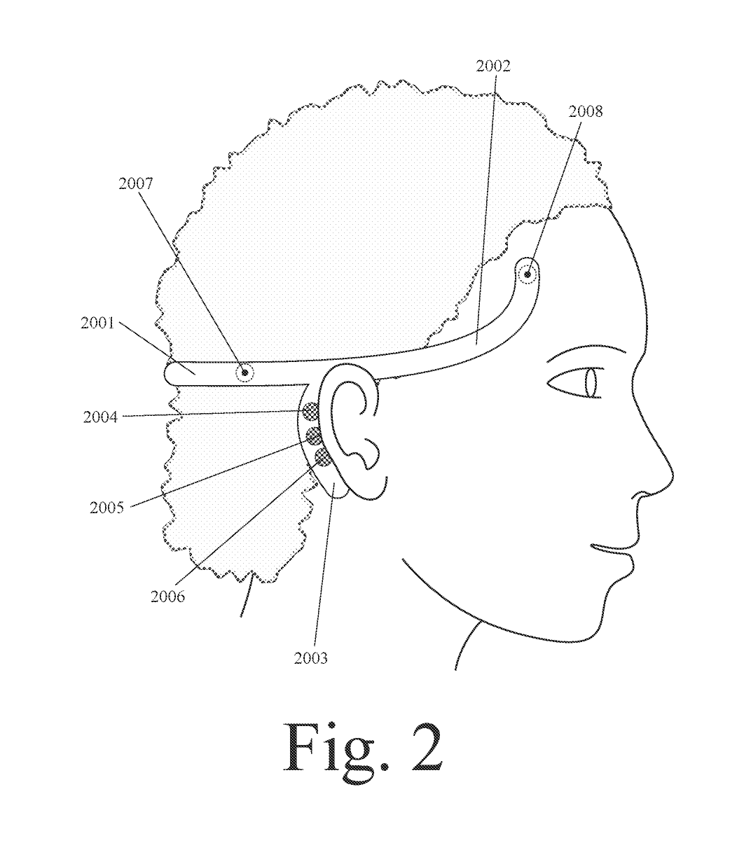

FIG. 2 shows an example of a wearable device for measuring electromagnetic brain activity comprising: a partially-circumferential headband (including rear portion 2001, front portion 2002, and ear-perimeter-engaging member 2003) which spans a portion of the circumference of a person's head, including a portion of the person's forehead; a plurality of electromagnetic energy sensors (including 2007 and 2008) which are configured to be held in proximity to the person's head by the headband, wherein these electromagnetic energy sensors collect data concerning electromagnetic activity of the person's brain; a wireless data transmitter and/or receiver 2004; a data processor 2005; and a power source 2006. In an example, this device can have a symmetric configuration on the other side of the person's head, which is not shown here.

In an example, a rear portion of a partially-circumferential headband can extend rearward from a person's right and left ears, looping completely around the rear of a person's head from the right ear to the left ear. In an example, right and left front portions of a partially-circumferential headband can extend forward from a person's right and left ears, respectively, partially extending onto the right and left sides of a person's forehead, respectively, but not completely spanning from the right ear to the left ear. In an example, the right and left front portions of a partially-circumferential headband can have ends which terminate on the right and left sides of a person's forehead, respectively, leaving a gap between them. In an example, this gap can include the center of the person's forehead.

In an example, a partially-circumferential headband can span between 50% and 85% of the circumference of a person's head. In an example, a partially-circumferential headband can span between 60% and 80% of the circumference of a person's head. In an example, a partially-circumferential headband can have an arcuate axial shape like that of an ancient Roman laurel wreath. In an example, a partially-circumferential headband can be shaped like a horseshoe or like the letter “U”, with upturned front ends. In an example, a partially-circumferential headband can loop around the sides and rear of a person's head from the right side of a person's forehead to the left side of the person's forehead, but not fully span across the person's forehead. In an example, a partially-circumferential headband can fully span the rear of a person's head, between their ears, but only partially span the front of the person's head.

In an example, a partially-circumferential headband can rest on top of a person's ears. In an example, a partially-circumferential headband can span the sides of a person's head above the person's ears. In an example, a partially-circumferential headband can loop around the rear of a person's head at a substantially level height, pass over the tops of a person's ears, and then arc upwards and forward to terminal positions on the sides of the person's forehead, stopping short of the center of the person's forehead. In an example, the right and left ends of a partially-circumferential headband can be on a person's forehead above the person's right and left eyes, respectively.

In an example, a side of a partially-circumferential headband can extend forward from a person's ear at an overall vector between the 1 o'clock (30 degree) vector and the 3 o'clock (90 degree) vector. In an example, a front portion of a partially-circumferential headband can initially extend forward from a person's ear along a vector between the 2 o'clock (60 degree) and 3 o'clock (90 degree) vectors, and then curve upward toward the person's forehead along a vector between the 1 o'clock (30 degree) and 2 o'clock (60 degree) vectors. In an example, a front portion of a partially-circumferential headband can be configured to end between 25% and 75% of the way from a person's ear to the center of their forehead. In an example, this end can be within the range of 1″ to 4″ above the top of the person's ear.

In an example, this headband can further comprise an ear-perimeter-engaging member which curves around the rear of a person's ear to better hold the headband in place. In an example, this ear-perimeter-engaging member can span between the 7 o'clock (210 degree) and 12 o'clock (0 degree) vectors. In an example, this ear-perimeter-engaging member can span between the 9 o'clock (270 degree) and 12 o'clock (0 degree) vectors. In an example, this ear-perimeter-engaging member can also be attached to an earlobe.

FIG. 2 also shows an example of a wearable brain activity monitoring device comprising: a plurality of brain activity sensors (including 2007 and 2008); a head-worn loop (including rear portion 2001 and front portion 2002) which is configured to span from one ear to the other around the lower-posterior portion of the head of a person wearing the loop, wherein the loop is configured to position the plurality brain activity sensors at selected locations on the person's head; a wireless data transmitter and/or receiver 2004; a data processor 2005; and a power source 2006. Relevant embodiment variations discussed elsewhere in this disclosure or in other disclosures which are incorporated by reference in the priority claims can be applied to the example shown here in this figure.

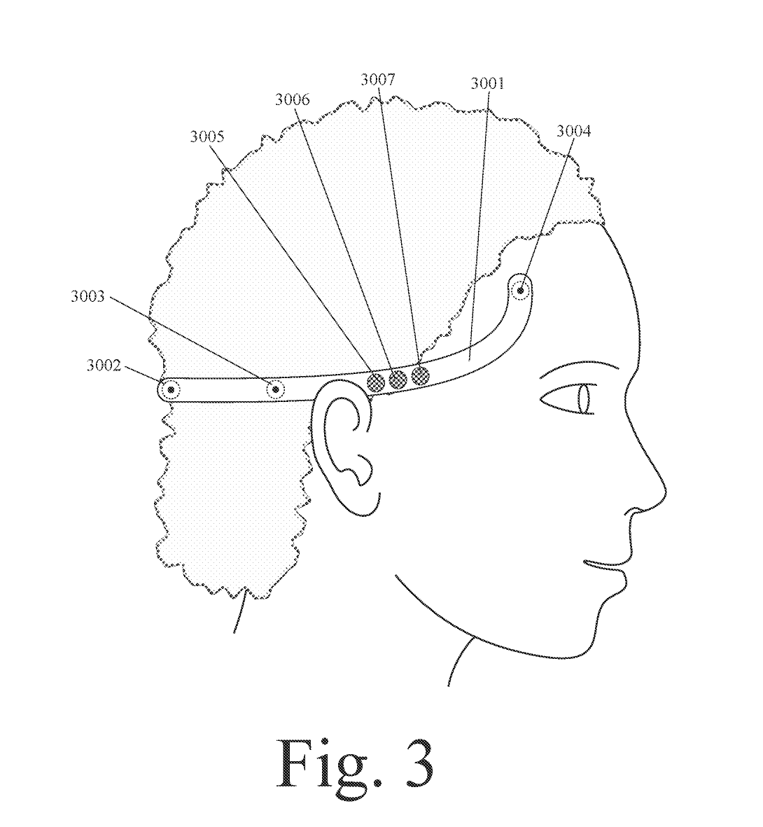

FIG. 3 shows an example of a wearable brain activity monitoring device comprising: a plurality of brain activity sensors (including 3002, 3003, and 3004); a head-worn loop 3001 which is configured to span from one ear to the other around the lower-posterior portion of the head of a person wearing the loop, wherein the loop is configured to position the plurality brain activity sensors at selected locations on the person's head; a wireless data transmitter and/or receiver 3004; a data processor 3005; and a power source 3006. Relevant embodiment variations discussed elsewhere in this disclosure or in other disclosures which are incorporated by reference in the priority claims can be applied to the example shown here in this figure.

In an example, this invention can be embodied in a wearable brain activity monitoring device comprising: a plurality of brain activity sensors; a head-worn loop which is configured to span from one ear to the other around the lower-posterior portion of the head of a person wearing the loop, wherein this loop is configured to position the plurality of brain activity sensors at selected locations on the person's head; a data processing component; and a power source or transducer. In an example, the brain activity sensors can be electrodes.

In an example, the plurality of brain activity sensors can comprise six brain activity sensors. In an example, the selected locations of brain activity sensors can be F3, F4, P3, P4, O1, and O2. In an example, the selected locations of brain activity sensors can be T3 or T7, T4 or T8, T5 or P7, T6 or P8, O1, and O2. In an example, a plurality of brain activity sensors can comprise four brain activity sensors. In an example, the selected locations of brain activity sensors can be F3, F4, O1, and O2. In an example, the average height of a head-worn loop can be configured to be equal to, or lower than, the average height of a person's ears. In an example, ends of a head-worn loop can be configured to terminate at locations which are forward of a person's ears.

In an example, this invention can be embodied in a wearable brain activity monitoring device comprising: a partially-circumferential headband, wherein the partially-circumferential headband spans a portion of the circumference of a person's head, including a portion of the person's forehead; a plurality of electromagnetic energy sensors which are configured to be held in proximity to the person's head by the partially-circumferential headband, wherein these electromagnetic energy sensors collect data concerning electromagnetic activity of the person's brain; a wireless data transmitter and/or receiver; a data processor; and a power source.

In an example, electromagnetic energy sensors can be electrodes. In an example, a plurality of electromagnetic energy sensors can comprise six electromagnetic energy sensors. In an example, the selected locations of electromagnetic energy sensors can be F3, F4, P3, P4, O1, and O2. In an example, the selected locations of electromagnetic energy sensors can be T3 or T7, T4 or T8, T5 or P7, T6 or P8, O1, and O2. In an example, the average height of a partially-circumferential headband can be configured to be equal to, or lower than, the average height of a person's ears. In an example, ends of a partially-circumferential headband can be configured to terminate at locations which are forward of a person's ears.

In an example, this invention can be embodied in a wearable brain activity monitoring device comprising: a plurality of brain activity sensors; a head-worn loop which is configured to span from one ear to the other around the lower-posterior portion of the head of a person wearing the loop, wherein this loop is configured to position the plurality of brain activity sensors at selected locations on the person's head; a data processing component; and a power source or transducer. In an example, the plurality of brain activity sensors comprises four brain activity sensors. In an example, the selected locations of brain activity sensors are F3, F4, O1, and O2. In an example, ends of the head-worn loop are configured to terminate at locations which are forward of the person's ears.

In an example, a wearable brain activity monitoring device can be worn on a person's head, spanning from a right side of the person's forehead to the left side of the person's forehead, around the lower-posterior surface of the person's head, and resting on top of the person's ears. In an example, when the person is standing up with their head erect, the portions of this device which are on the person's forehead are a first average height and the portions of this device which span the lower-posterior surface of the person's head are a second average height, wherein the first average height is higher than the second average height.

In an example, a wearable brain activity monitoring device can further comprise a loop which encircles a person's ear to help hold the device in place on a person's head. In an example, this loop can be elastic and/or stretchable. In an example, a wearable brain activity can further comprise a clip, clasp, clamp, snap, hook, or other attachment mechanism by which it can be removably attached to eyewear. In an example, a wearable brain activity can further comprise a clip, clasp, clamp, snap, hook, or other attachment mechanism by which it can be removably attached to an eyeglass frame. In an example, a wearable brain activity monitoring device can further comprise a set of moose antlers. In an example, a wearable brain activity monitoring device can have telescoping ends which reversibly extend forward and/or upward from a person's ears to positions on the person's forehead. In an example, a wearable brain activity monitoring device can have pivoting, tilting, rotating, and/or folding ends which reversibly extend forward and/or upward from the sides of a person's head to positions on the person's forehead.

In an example, a device does not completely encircle the person's head. In an example, there is a gap between ends of a device on the sides of the person's forehead. In an example, this gap can be in the range of 3″ to 8″. In an example, a device can completely encircle a person's head, but a portion of the device can be elastic and/or stretchable. In an example, an elastic and/or stretchable portion of a device can span a center portion of the person's forehead. In an example, an elastic and/or stretchable portion of the device can have a length in the range of 3″ to 9″. In an example, a device can completely encircle the person's head, but at least a portion of the device can be transparent or translucent. In an example, a transparent or translucent portion of a device can span a center portion of the person's forehead. In an example, a transparent or translucent portion of a device can have a length in the range of 3″ to 9″.

In an example, a wearable brain activity monitoring device can further comprise one or more actuators whose activation changes the fit of the device—such as by changing the proximity, pressure, force, and/or elasticity between the device and the surface of a person's head. In an example, the fit of the device can be manually adjusted. In an example, the fit of the device can be automatically adjusted by one or more actuators in response to a person's movement and/or acceleration. In an example, one or more actuators can make the device fit more tightly against the surface of the person's head when the person is engaged in more vigorous movement and/or rapid acceleration. In an example, the device can hold onto a person's head more tightly when the person is moving quickly. In an example, the fit of the device can be automatically adjusted by one or more actuators in response to a person's body configuration or orientation. In an example, one or more actuators can make the device fit more tightly against the surface of the person's head when the person head's head is oriented sideways or upside-down.

In an example, a wearable brain activity monitoring device can further comprise a camera. In an example, a camera can be configured to be activated when a person creates of a selected pattern of electromagnetic brain activity. In an example, the focal direction and/or focal distance of the camera can be changed when a person creates different patterns of electromagnetic brain activity. In an example, a wearable brain activity monitoring device can further comprise a speaker and/or computerized voice generator. In an example, a computerized voice generator can generate selected words (which are emitted by a speaker) when a person creates selected patterns of electromagnetic brain activity. In an example, this device can be part of a system which further comprises a database of selected words associated with selected patterns of electromagnetic brain activity, enabling a person to communicate selected words by creating selected patterns of electromagnetic activity with their mind.

In an example, this invention can be embodied in a wearable brain activity monitoring device with a plurality of electromagnetic brain activity sensors which are held in place at selected locations on a person's head by a partially-circumferential headband . The partially-circumferential headband can curve around the lower-posterior surface of a person's head, from one ear to the other, and have forward ends which extend upward from the person's ears to the sides of the person's forehead. In an example, the device can have six brain activity sensors which are located at the F3, F4, P3, P4, O1, and O2 standard EEG sensor locations.

I claim:

1. A wearable brain activity monitoring device comprising:
a plurality of brain activity sensors;
a head-worn loop which is configured to span from one ear to the other around the lower-posterior portion of the head of a person wearing the loop, wherein this loop is configured to position the plurality of brain activity sensors at selected locations on the person's head;
a data processing component; and
a power source or transducer.

2. The device in claim 1 wherein the brain activity sensors are electrodes.

3. The device in claim 1 wherein the plurality of brain activity sensors comprises six brain activity sensors.

4. The device in claim 3 wherein the selected locations of brain activity sensors are F3, F4, P3, P4, O1, and O2.

5. The device in claim 3 wherein the selected locations of brain activity sensors are T3 or T7, T4 or T8, T5 or P7, T6 or P8, O1, and O2.

6. The device in claim 1 wherein the plurality of brain activity sensors comprises four brain activity sensors.

7. The device in claim 6 wherein the selected locations of brain activity sensors are F3, F4, O1, and O2.

8. The device in claim 1 wherein the average height of the head-worn loop is configured to be equal to, or lower than, the average height of the person's ears.

9. The device in claim 1 wherein ends of the head-worn loop are configured to terminate at locations which are forward of the person's ears.

10. A wearable brain activity monitoring device comprising:
a partially-circumferential headband, wherein the partially-circumferential headband spans a portion of the circumference of a person's head, including the lower-posterior surface of the person's head and a portion of the person's forehead;
a plurality of electromagnetic energy sensors which are configured to be held in proximity to the person's head by the partially-circumferential headband, wherein these electromagnetic energy sensors collect data concerning electromagnetic activity of the person's brain;
a wireless data transmitter and/or receiver;
a data processor; and
a power source.

11. The device in claim 10 wherein the electromagnetic energy sensors are electrodes.

12. The device in claim 10 wherein the plurality of electromagnetic energy sensors comprises six electromagnetic energy sensors.

13. The device in claim 10 wherein the selected locations of electromagnetic energy sensors are F3, F4, P3, P4, O1, and O2.

14. The device in claim 10 wherein the selected locations of electromagnetic energy sensors are T3 or T7, T4 or T8, T5 or P7, T6 or P8, O1, and O2.

15. The device in claim 10 wherein the average height of the partially-circumferential headband is configured to be equal to, or lower than, the average height of the person's ears.

16. The device in claim 10 wherein ends of the partially-circumferential headband are configured to terminate at locations which are forward of the person's ears.

17. A wearable brain activity monitoring device comprising:
a plurality of brain activity sensors;
a head-worn loop which is configured to span from one ear to the other around the lower-posterior portion of the head of a person wearing the loop, wherein this loop is configured to position the plurality of brain activity sensors at selected locations on the person's head;
a data processing component; and
a power source or transducer.

18. The device in claim 17 wherein the plurality of brain activity sensors comprises four brain activity sensors.

19. The device in claim 17 wherein the selected locations of brain activity sensors are F3, F4, O1, and O2.

20. The device in claim 17 wherein ends of the head-worn loop are configured to terminate at locations which are forward of the person's ears.
